(19) United States
(12) Patent Application Publication
(10) Pub. No.: US 2024/0183794 A1
(43) Pub. Date: Jun. 6, 2024

(54) Composite Component Artifact Detection

(71) Applicant: McLaren Automotive Limited, Woking (GB)

(72) Inventor: Phillip Kearney, Guilford (GB)

(21) Appl. No.: 18/287,852

(22) PCT Filed: Apr. 22, 2022
(86) PCT No.: PCT/GB22/51032

§ 371 Date: Oct. 20, 2023

(30) Foreign Application Priority Data
Apr. 22, 2021 (GB) 2105768.2

(51) Int. Cl. G01N 21/95; G01B 11/06

(52) U.S. Cl. CPC G01N 21/95; G01B 11/06

(57) ABSTRACT

A method for detecting artifacts in a surface of a composite component, the composite component comprising reinforcement material, matrix material interspersed with the reinforcement material and matrix material forming the surface of the composite component, the method comprising: directing incident light on to the surface of the composite component, the incident light having a first wavelength; recording radiated light originating from positions on the surface, the radiated light being generated in response to the incident light being directed on to the surface and the radiated light having respective second wavelengths for each position; determining a surface thickness for each position on the surface based on the second wavelength of the radiated light for that position; and detecting one or more artifacts in the matrix material forming the surface based on the surface thickness of at least one position on the surface matching at least one artifact criteria.

### CROSS-REFERENCE TO RELATED APPLICATIONS

The present application is the national stage entry of International Patent Application No. PCT/GB2022/051032, filed on Apr. 22, 2022, and claims priority to GB Application No. GB2105768.2, filed on Apr. 22, 2021, the disclosures of which are incorporated herein by reference thereto.

### FIELD OF THE INVENTION

This invention relates to a method and system for detecting artifacts in a surface of a composite material.

### BACKGROUND

It is known to manufacture components from fibre-reinforced composite (FRC) materials. Such materials typically comprise a matrix that contains reinforcing fibres. As an example, the matrix could be an epoxy resin and the fibres could be carbon fibre (CF) strands. Materials of this type can have good strength in comparison to their weight. However, the processes required to make components from fibre-reinforced materials can be complex. The strength of the components made with FRC materials are dependent on the reinforcing fibres running in the correct directions inside the component and the matrix material being dispersed in a controlled manner between and around the reinforcing fibres.

One process for forming FRC components is resin transfer moulding (RTM). In this process the reinforcing fibres are laid up in a mould cavity, liquid resin is injected into the mould cavity and the resin is cured, typically by heating the mould body. Once the resin has become solid the mould can be opened and the resulting component removed. The resin can be injected by drawing a vacuum in the mould cavity and allowing the vacuum to pull the resin into the mould. The resin can also be injected by depositing the resin into the mould cavity whilst the mould is open and then closing the mould on to the resin. Excess resin is squeezed out of the mould during the closing of the mould and/or excess resin can be collected in appropriately designed flash areas adjacent to the reinforcing fibres in such a way that such flash area can be trimmed off the moulded component after moulding. The mould cavity can be defined by rigid mould tools, which has the advantage of giving good control over the dimensional accuracy and surface finish of the component. Furthermore, long fibre runs, and woven mats of fibres can be embedded in the matrix, giving the end component great strength. RTM can be used for major structural components, such as vehicle tubs, as described in EP 2 772 416.

Another process for forming FRC components uses reinforcing fibres that are pre-impregnated with a matrix material such as a resin. These are generally known as pre-preg. These reinforcing fibres can be laid up in a mould cavity and then the pre-impregnated matrix material can be cured, typically by heating the mould body. The heating may take place in an autoclave. Once the resin has become solid the mould can be opened and the resulting component removed.

In each method for forming FRC components, the resulting component is inspected to ensure that the moulding process was completed successfully so that the component has the required physical properties. For instance, the component may be a structural part of a vehicle and so need to endure a level of force in particular directions. One area of the moulded component that is inspected is the surface information of the composite structure. The surface of the component generally has matrix material which encapsulates the reinforcement material inside. The surface matrix material is important as it ensures that the reinforcement material does not splinter near the surface and also assists in directing the forces on the component along the desired routes through the component. Artifacts present in the surface matrix material can therefore affect the strength of the composite component.

Traditional inspection techniques rely on techniques such as thermography, CT-scanning, ultrasonic scanning and visual inspections. These processes are very time consuming, expensive and require a highly skilled operative to undertake and interpret the results of the equipment. These traditional methods are less than optimal due to the length of time it takes to inspect a component and the reliance on particular skilled operatives to undertake the inspections.

It would therefore be desirable for there to be an improved method of detecting artifacts in a surface of a composite component.

### SUMMARY

According to a first aspect of the present invention there is provided a method for detecting artifacts in a surface of a composite component, the composite component comprising reinforcement material, matrix material interspersed with the reinforcement material and matrix material forming the surface of the composite component, the method comprising: directing incident light on to the surface of the composite component, the incident light having a first wavelength; recording radiated light originating from positions on the surface, the radiated light being generated in response to the incident light being directed on to the surface and the radiated light having respective second wavelengths for each position; determining a surface thickness for each position on the surface based on the second wavelength of the radiated light for that position; and detecting one or more artifacts in the matrix material forming the surface based on the surface thickness of at least one position on the surface matching at least one artifact criteria.

The incident light may be ultraviolet light. The first wavelength may be between 10 nm and 400 nm. The first wavelength may be between 315 nm and 400 nm.

Recording radiated light originating from positions on the surface may comprise recording one or more images of the surface using an image recording device. The radiated light may be generated by scattering of the incident light within the surface of the composite component.

Determining a surface thickness for each position on the surface may be based on determining a difference between the first wavelength and the second wavelength of the radiated light for that position. Determining a surface thickness for each position on the surface may comprise comparing the difference between the first wavelength and the second wavelength of the radiated light for that position to a line of best fit of surface thickness against light wavelength differences. Determining a surface thickness for each position on the surface may comprise comparing the second wavelength to a line of best fit of surface thickness against radiated light wavelengths. Determining a surface thickness for each position on the surface may comprise selecting the surface thickness from the line of best fit that has a radiated light wavelength that matches the second wavelength.

The artifact criteria may comprise the surface thickness being less than a threshold thickness, and detecting one or more artifacts may comprise detecting an artifact when the surface thickness is less than the threshold thickness. The artifact criteria may comprise a region of positions having a surface thickness being less than a threshold thickness, and detecting one or more artifacts may comprise detecting an artifact when a region of surface thicknesses is less than the threshold thickness.

The artifact criteria may comprise a region of positions having a surface thickness being less than a threshold thickness surrounded by a region of positions that have a surface thickness greater than an expected thickness, and detecting one or more artifacts may comprise detecting an artifact when a region of positions having a surface thickness being less than a threshold thickness is surrounded by a region of positions that have a surface thickness greater than an expected thickness.

According to a second aspect of the present invention there is provided a system for detecting artifacts in a surface of a composite component, the composite component comprising reinforcement material, matrix material interspersed with the reinforcement material and matrix material forming the surface of the composite component, the system comprising: a lamp configured to direct incident light on to the surface of the composite component, the incident light having a first wavelength; an image recording device configured to record radiated light originating from positions on the surface, the radiated light being generated in response to the incident light being directed on to the surface and the radiated light having respective second wavelengths for each position; and a processor coupled to the image recording device to receive second wavelength measurements and being configured to: determine a surface thickness for each position on the surface based on the second wavelength of the radiated light for that position; and detect one or more artifacts in the matrix material forming the surface based on the surface thickness of at least one position on the surface matching at least one artifact criteria.

### BRIEF DESCRIPTION OF THE FIGURES

The present invention will now be described by way of example with reference to the accompanying drawings. In the drawings:

FIG. 1 shows an example composite component.

FIG. 2 shows a cut through view of the example composite component.

FIGS. 3a, b, c, d, e shows a pictorial process flow diagram.

### DETAILED DESCRIPTION

The following description is presented to enable any person skilled in the art to make and use the invention, and is provided in the context of a particular application. Various modifications to the disclosed embodiments will be readily apparent to those skilled in the art.

The general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

The present invention relates to a method for detecting artifacts in a surface of a composite component, the composite component comprising reinforcement material, matrix material interspersed with the reinforcement material and matrix material forming the surface of the composite component. The method comprises directing incident light on to the surface of the composite component, the incident light having a first wavelength. The method further comprises recording radiated light originating from positions on the surface, the radiated light being generated in response to the incident light being directed on to the surface and the radiated light having respective second wavelengths for each position. The method further comprises determining a surface thickness for each position on the surface based on the second wavelength of the radiated light for that position; and detecting one or more artifacts in the matrix material forming the surface based on the surface thickness of at least one position on the surface matching at least one artifact criteria.

The present invention also relates to a system for detecting artifacts in a surface of a composite component, the composite component comprising reinforcement material, matrix material interspersed with the reinforcement material and matrix material forming the surface of the composite component. The system comprises a lamp configured to direct incident light on to the surface of the composite component, the incident light having a first wavelength. The system also comprises an image recording device configured to record radiated light originating from positions on the surface, the radiated light being generated in response to the incident light being directed on to the surface and the radiated light having respective second wavelengths for each position. The system also comprises a processor coupled to the image recording device to receive second wavelength measurements and being configured to: determine a surface thickness for each position on the surface based on the second wavelength of the radiated light for that position; and detect one or more artifacts in the matrix material forming the surface based on the surface thickness of at least one position on the surface matching at least one artifact criteria.

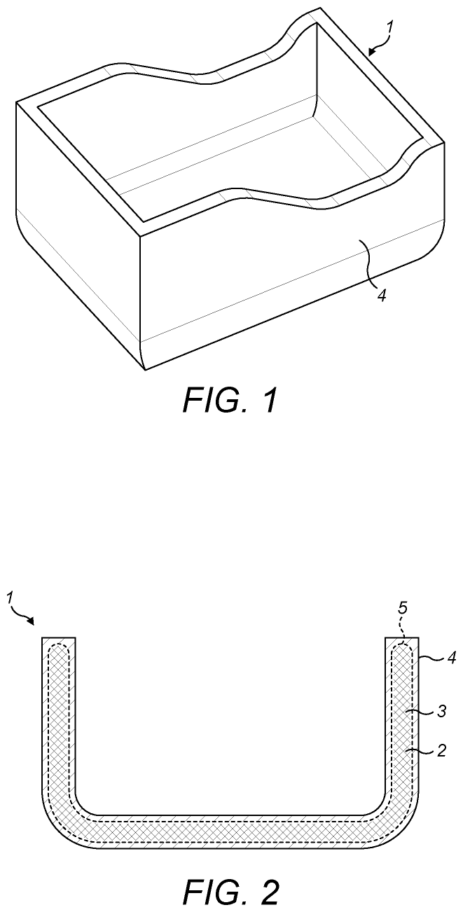

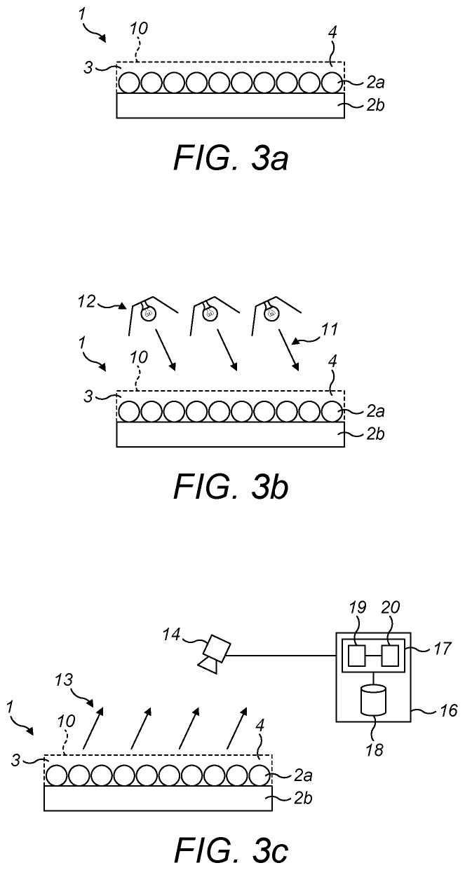

FIG. 1 shows an example composite component 1. FIG. 2 shows a cut through view of the composite component 1 to show the structure that the composite component is formed from. It will be appreciated that the FIGS. 1 and 2 are schematic views. In particular, FIG. 2 shows a schematic view of the formation of the composite component and is not intended to be to scale. The composite component comprises reinforcement material 2 and matrix material 3. The matrix material 3 is interspersed with the reinforcement material 2. In this way, the reinforcement material 2 has been infused with the matrix material 3. The matrix material 3 holds the reinforcement material 2 in place to provide the structure of the composite component. The matrix material 3 may have been solidified through a process such as curing. The curing may be undertaken by subjecting the matrix material to radiation, such as UV radiation, or to heat.

The composite component 1 has a surface 4. This surface 4 forms the external surface of the composite component 1. The external surface being the outer surface of the composite component 1. As shown in FIG. 2, the matrix material 3 forms the surface 4 of the composite component 1. The matrix material 3 forms a layer over the reinforcement material 2 which forms the surface of the composite component 1. This layer is shown in FIG. 2 by dotted line 5 which delimits the reinforcement material 2 that is infused with the matrix material 3 from the surface region of the composite component 1.

The reinforcement material 2 may be any reinforcement that is suitable for infusion with a matrix material. The reinforcement material may comprise carbon fibre. The reinforcement material may be carbon fibre. The reinforcement material may be weaved into mats. The reinforcement material may be held together by threads that are sewn through the reinforcement material. In this way, the reinforcement material may be held together by stitching.

The matrix material may comprise a resin. The matrix material may comprise an epoxy resin. A hardener may have been used to assist in the curing of the resin. Therefore, the matrix material may incorporate the hardener. The hardener may be an amine-based hardener.

Other parts may be part of the composite component. For instance, the composite component may comprise one or more of metal parts or foam cores. Metal parts may be bonded to the reinforcement material 2 by the matrix material 3. Foam cores could be bonded to the reinforcement material 2 by the matrix material 3.

It is important that the matrix material 3 that forms the surface of the composite component 1 has the correct thickness. This is so that the composite component 1 has the required strength over the entirety of the composite component 1. Due to the forming techniques used to form the composite component, such as RTM or cured pre-preg as described herein, there is a risk that the matrix material 3 does not flow through and over the reinforcement material 2 in completely the desired manner. This can lead to artifacts in the composite component. These artifacts may be described as forming artifacts as they arise during the forming of the composite component. The artifacts may be inconsistencies in the formation of the matrix material 3. The artifacts may be deviations from the desired form of the matrix material 3 in the composite component. The artifacts may be present in the surface 4 of the composite component 1. I.e. the layer of matrix material 3 that forms the surface 4 of the composite component.

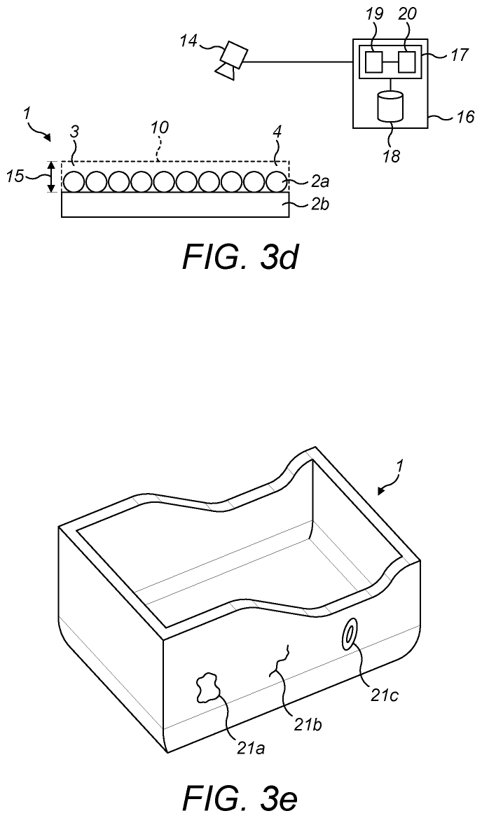

The improved method by which artifacts in the surface of the composite component can be detected will now be described with reference to FIG. 3. FIG. 3 shows a pictorial process flow diagram of the method steps.

FIG. 3a shows a portion of the composite component 1. Portion shows the surface 4 together with the matrix material 3 which is shown delimited by dashed line 10. The portion also shows the reinforcement material 2 present in the portion. In this example two layers of reinforcement material 2 are shown at 2a and 2b. The fibres of a first layer 2a of reinforcement material 2 are shown as running in one direction and the fibres of a second layer 2b of reinforcement material 2 are shown as running in a different direction to the first layer 2a. The surface layer is shown as comprising the matrix material 3 which is formed around the reinforcement material 2. In this way the matrix material 3 encapsulates the reinforcement material 2.

Therefore, in FIG. 3a a composite component 1 is provided. The composite component 1 comprising reinforcement material 2 and matrix material 3 interspersed with the reinforcement material 2. The matrix material 3 bonding the reinforcement material 2 together. The matrix material 3 forms a surface 4 of the composite component.

FIG. 3b shows incident light 11 being directed on to the surface 4 of the composite component 1. The incident light 11 is generated by one or more lamps 12. The lamps 12 are positioned to direct the incident light 11 on to the surface 4. There may be sufficient lamps 12 that the entire surface of the composite component can be illuminated at the same time. In this way, the incident light 11 may be directed onto the whole surface of the composite component 1 at the same time. There may only be enough lamps 11 to illuminate part of the surface of the composite component 1 at any given moment. The lamps 12 may be moveable to permit the entire surface of the composite component 1 to be illuminated in turn. Alternatively, the composite component 1 may be loaded onto a carriage which permits the composite component 1 to be moved relative to the lamps 11 to enable the illumination of the entire surface of the composite component 1 in turn. In this way, the incident light 11 may be directed onto the whole surface of the composite component 1 in turn. It will be understood that only particular regions of the composite component 1 may be important for the structural integrity of the component. In these cases, it may be decided to only analyse in a portion of the surface of the composite component 1. Therefore, a portion of the surface of the composite component 1 may be subjected to illumination by the incident light 11.

The incident light has a first wavelength. The incident light may be ultraviolet light. It has been found that it is advantageous to use ultraviolet light as when it travels through particular matrix materials the matrix materials may then emit light in the visible spectrum. The emission of visible spectrum light makes it easier to capture by standard image capture devices such as cameras. In other examples, the light that is emitted by the matrix materials may be in a non-visible spectrum of light or may be a spectrum that incorporates both visible and non-visible light. In these cases, image capture device(s) may be used that can capture the relevant spectrum of light. The first wavelength may be between 10 nm and 400 nm. Preferably, the first wavelength is between 315 nm and 400 nm. More preferably, the first wavelength is between 325 nm and 375 nm. The wavelength of the incident light may be selected based on the matrix material and the desired wavelength range at which it is required that the matrix material emit light at. It will be appreciated that the lamps that produce the light may not be perfect radiators at a particular wavelength. Therefore, when the incident light is described as having a first wavelength this may be interpreted as having a spectrum that is centred on that wavelength. In this way, the incident light is predominantly constituted by light of the first wavelength.

FIG. 3c shows radiated light 13 being emitted by the matrix material 4. The radiated light is recorded originating from positions on the surface of the composite component 1. The recording may be undertaken by one or more image recording devices 14. The image recording device 14 may be a camera. One or more images may be generated which record the wavelength of light emanating from each position on the surface of the composite component 1. The recording of the radiated light 13 may include the wavelength of the radiated light for respective positions. The recording of the radiated light may include the intensity of the radiated light for respective positions. The radiated light 13 is recorded by one or more image recording devices 14. The image recording device(s) 14 are positioned to record the radiated light 13 emitted from the surface 4. The image recording device(s) 14 may capture a sufficient area that the entire surface of the composite component can be recorded at the same time. In this way, the radiated light 14 emitted from the whole surface may be captured at the same time. The image recording device(s) 14 may only be able to capture light from part of the surface of the composite component 1 at any given moment. The image recording device(s) 14 may be moveable to permit the entire surface of the composite component 1 to be imaged in turn. Alternatively, the composite component 1 may be loaded onto a carriage which permits the composite component 1 to be moved relative to the image recording device(s) 14 to enable the capture of the light emitted by the entire surface of the composite component 1 in turn. In this way, the radiated light 13 may be captured from the whole surface of the composite component 1 in turn. It will be understood that only particular regions of the composite component 1 may be important for the structural integrity of the component. In these cases, it may be decided to only analyse in a portion of the surface of the composite component 1. Therefore, a portion of the surface of the composite component 1 may have the radiated light recorded from it.

The radiated light 13 has respective second wavelengths for each position. The radiated light from multiple positions may have the same wavelength whilst other positions may have different wavelengths. In this way, different positions of the surface of the composite component may radiate different colours relative to other positions.

The radiated light 13 is generated in response to the incident light 11 being directed on to the surface 4 of the composite component. The incident light 11 passes into the composite component 1. Some of the incident light may be absorbed by the reinforcement material. Only a small percentage of the incident light may be reflected by the reinforcement material. This makes the reinforcement material itself appear dark in the recorded images of the composite component. The remainder of the incident light may be scattered by the matrix material. The scattering of the incident light causes a change in wavelength of the incident light which causes the light radiated by the composite component 1 and, in particular, the matrix material to be at different wavelengths to that of the incident light 11. During the scattering of the incident light, the matrix material absorbs some of the energy of the incident light 11 which causes the change in wavelength of the incident light. The thicker the surface layer of matrix material, the more energy that will be absorbed from the incident light 11. In this way, the thickness of the matrix material at a particular position of the surface of the composite component can be inferred. As energy is absorbed in the matrix material from the incident light, the second wavelengths are generally longer than the first wavelength.

As shown in FIG. 3d, the surface thickness 15 is determined for each position on the surface 4 of the composite component 1 that is emitting the radiated light 13. The surface thickness 15 is determined based on the second wavelength of the radiated light 13 for that position. It will be appreciated that each position on the surface of the composite component may not be perfect radiators at a particular wavelength. Therefore, when the radiated light is described as having a second wavelength this may be interpreted as having a spectrum that is centred on that wavelength. In this way, the radiated light is predominantly constituted by light of the second wavelength. The method may comprise determining a central wavelength of the radiated light as the second wavelength of the radiated light.

The difference between the first wavelength and the second wavelength of the radiated light 13 for that position may be determined. The difference may be used to determine the surface thickness 15 for that position. Alternatively, the second wavelength of the radiated light 13 may be used directly.

The surface thickness 15 may be determined by a processing device 16. Processing device 16 may be connected to the image recording device 14 to receive images captured by the image recording device 14. Where more than one image recording device 14 is present then it will be appreciated that each image recording device 14 may be connected to the processing device 16. The images may be sent to processing device 16 from image recording device(s) 14.

It will be appreciated that, whilst reference is made to a discrete device with reference to processing device 16, this device could be part of a cluster of devices or may be a virtual server running in a cloud-based, virtual environment. The processing device 16 comprises a processing section 17 and a storage section 18. The processing device 16 is configured to implement the methods described herein for processing radiated light from a composite component and determining artifacts. These methods can be implemented and controlled by the processing section 17. The processing section 17 could perform its methods using dedicated hardware, using a general purpose processor executing software code, or using a combination of the two. A processor 19 executed software code stored in a non-transient way in software memory 20 in order to perform its methods. The processing section can read/write data from/to storage location 18. The storage location 3 may be in the form of a memory. Storage location 3 may comprise non-volatile memory, may be in the form of an array of discrete banks or memory such as hard disks. Whilst shown in FIG. 3c as schematically being part of the processing device 16, the storage location 18 may be separate from the processing device 16 and connected to the processing device 16. The storage location may comprise one or more look up tables, lines of best fit and other data used as part of the methods described herein.

As described herein, it has been determined that the wavelength with which a position of the surface region 4 of the composite component 1 radiates light corresponds with the thickness of the surface layer of matrix material 3. Therefore, the surface thickness 15 for each position can be determined from the second wavelength of the radiated light from that position.

The surface thickness 15 may be determined by comparing the second wavelength to a line of best fit of surface thickness against radiated light wavelengths. The line of best fit may correlate radiated light wavelengths to surface thickness. There may be one surface thickness per radiated light wavelength. The line of best fit may be a continuous line over the wavelengths with which the surface may radiate with. The line of best fit may be dependent on the wavelength of the incident light. Therefore, determining the surface thickness 15 may comprise selecting the line of best fit for the first wavelength of incident light.

The surface thickness 15 may be determined by comparing the difference between the first wavelength and the second wavelength to a line of best fit of surface thickness against light wavelength differences. The line of best fit may correlate light wavelength differences to surface thickness. There may be one surface thickness per light wavelength difference. The line of best fit may be a continuous line over the wavelength differences with which the surface may radiate with. The line of best fit may be dependent on the wavelength of the incident light. Therefore, determining the surface thickness 15 may comprise selecting the line of best fit for the first wavelength of incident light.

The step of determining a surface thickness for each position on the surface based on the second wavelength of the radiated light for that position may output a map of surface thickness over the surface of the composite component 1. The surface may be a portion of the whole surface of the composite component 1.

As shown in FIG. 3e, one or more artifacts 21 in the matrix material forming the surface are detected. The artifacts 21 are detected based on the surface thickness of at least one position on the surface matching at least one artifact criteria. The artifact criteria determine whether a surface thickness at a given position indicates that an artifact is present in the matrix material forming the surface. As described herein, the artifacts may be inconsistencies in the formation of the matrix material. The artifact criteria may be descriptors of surface thicknesses known artifacts that are compared to the surface thickness of the at least one position to determine whether an artifact exists. The descriptors may be maps of surface thickness of known artifacts.

The artifact criteria may define that an artifact exists if the surface thickness is below a threshold thickness. Therefore, if that artifact criteria is applied to a position on the surface and the position has a surface thickness less than the threshold value then an artifact will be determined as being present at that point. In this way, a position or region of low resin richness may be determined.

The artifact criteria may define that an artifact exists if a region of positions have a surface thickness below a threshold thickness. Therefore, if that artifact criteria is applied to a group of positions on the surface and the group of positions has a surface thickness less than the threshold value then an artifact will be determined as being present in that region. In this way, a region having a dry spot may be determined. A dry spot may be where matrix material did not fully wet the reinforcing material.

The artifact criteria may define that an artifact exists if a region of positions have a surface thickness below a threshold thickness that is surrounded by a region of positions that have a surface thickness above an expected thickness. The expected thickness may be the thickness of the surface region that is expected for a given composite component. Therefore, if that artifact criteria is applied to a group of positions on the surface and the group of positions has a region with surface thicknesses less than the threshold value that are surrounded by a region with surface thicknesses greater than an expected thickness then an artifact will be determined as being present in that region. In this way, a region having a surface void may be determined. A surface void may be an area inside the surface region that has separated to create a void inside the composite component.

The threshold thickness may be 3 mm, 2.5 mm, 2 mm, 1.5 mm, 1 mm, 0.5 mm, 0.3 mm or 0.1 mm. The expected thickness may be 0.1 mm, 0.3 mm, 0.5 mm, 1 mm, 2 mm, 2.5 mm, 3 mm, 3.5 mm or 4 mm.

The processing device 16 may apply the artifact criteria by comparing known artifacts which define the surface thickness of those artifacts to the surface thicknesses for each position. The processing device 16 may use a model which has been trained with a plurality of known artifacts. The known artifacts forming the artifact criteria. The processing device 16 can then apply the model to the surface thicknesses of each position to determine whether artifacts are present.

The output of the method may be a map of the detected artifacts relative to the surface of the composite component. The map may include all detected artifacts. Alternatively, the map may include those artifacts that are determined to be above a criticality threshold. The processing device 16 may output the map as a data file. The processing device 16 may output the map as an image or series of images that can be displayed on a display. The processing device 16 may comprise a display to output the image(s).

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A method for detecting artifacts in a surface of a composite component, the composite component comprising reinforcement material, matrix material interspersed with the reinforcement material and matrix material forming the surface of the composite component, the method comprising:
directing incident light on to the surface of the composite component, the incident light having a first wavelength;
recording radiated light originating from positions on the surface, the radiated light being generated in response to the incident light being directed on to the surface and the radiated light having respective second wavelengths for each position;
determining a surface thickness for each position on the surface based on the second wavelength of the radiated light for that position; and
detecting one or more artifacts in the matrix material forming the surface based on the surface thickness of at least one position on the surface matching at least one artifact criteria.

2. The method according to claim 1, wherein the incident light is ultraviolet light.

3. The method according to claim 1, wherein the first wavelength is between 10 nm and 400 nm.

4. The method according to claim 1, wherein the first wavelength is between 315 nm and 400 nm.

5. The method according to claim 1, wherein recording radiated light originating from positions on the surface comprises recording one or more images of the surface using an image recording device.

6. The method according to claim 1, wherein the radiated light is generated by scattering of the incident light within the surface of the composite component.

7. The method according to claim 1, wherein determining a surface thickness for each position on the surface is based on determining a difference between the first wavelength and the second wavelength of the radiated light for that position.

8. The method according to claim 7, wherein determining a surface thickness for each position on the surface comprises comparing the difference between the first wavelength and the second wavelength of the radiated light for that position to a line of best fit of surface thickness against light wavelength differences.

9. The method according to claim 1, wherein determining a surface thickness for each position on the surface comprises comparing the second wavelength to a line of best fit of surface thickness against radiated light wavelengths.

10. The method according to claim 9, wherein determining a surface thickness for each position on the surface comprises selecting the surface thickness from the line of best fit that has a radiated light wavelength that matches the second wavelength.

11. The method according to claim 1, wherein the artifact criteria comprise the surface thickness being less than a threshold thickness, and detecting one or more artifacts comprises detecting an artifact when the surface thickness is less than the threshold thickness.

12. The method according to claim 1, wherein the artifact criteria comprise a region of positions having a surface thickness being less than a threshold thickness, and detecting one or more artifacts comprises detecting an artifact when a region of surface thicknesses is less than the threshold thickness.

13. The method according to claim 1, wherein the artifact criteria comprise a region of positions having a surface thickness being less than a threshold thickness surrounded by a region of positions that have a surface thickness greater than an expected thickness, and detecting one or more artifacts comprises detecting an artifact when a region of positions having a surface thickness being less than a threshold thickness is surrounded by a region of positions that have a surface thickness greater than an expected thickness.

14. A system for detecting artifacts in a surface of a composite component, the composite component comprising reinforcement material, matrix material interspersed with the reinforcement material and matrix material forming the surface of the composite component, the system comprising:
a lamp configured to direct incident light on to the surface of the composite component, the incident light having a first wavelength;
an image recording device configured to record radiated light originating from positions on the surface, the radiated light being generated in response to the incident light being directed on to the surface and the radiated light having respective second wavelengths for each position; and
a processor coupled to the image recording device to receive second wavelength measurements and being configured to:
determine a surface thickness for each position on the surface based on the second wavelength of the radiated light for that position; and
detect one or more artifacts in the matrix material forming the surface based on the surface thickness of at least one position on the surface matching at least one artifact criteria.
